(19) United States
(12) Patent Application Publication
(10) Pub. No.: US 2008/0190855 A1
(43) Pub. Date: Aug. 14, 2008

(54) Derivatised Carbon

(75) Inventors: Richard Guy Compton, Oxford (GB); Gregory George Wildgoose, Oxford (GB)

Correspondence Address: KIRTON AND MCCONKIE, 60 EAST SOUTH TEMPLE,, SUITE 1800, SALT LAKE CITY, UT, 84111, US

(73) Assignee: ISIS INNOVATION LIMITED, Oxford (GB)

(21) Appl. No.: 11/913,762

(22) PCT Filed: May 5, 2006
(86) PCT No.: PCT/GB2006/001643

§ 371 Date: Jan. 28, 2008

(30) Foreign Application Priority Data
May 6, 2005 (GB) 0509307.5

(51) Int. Cl. C02F 1/28; C08G 69/26; C01B 31/02; C07D 487/00; C09C 1/56; C02F 1/62

(52) U.S. Cl. 210/688; 528/332; 527/207; 205/789.5; 532/1; 210/503

(57) ABSTRACT

Derivatised carbon is disclosed in which an amino acid or a derivative thereof is attached to the carbon. Derivatised carbon may be useful in the detection and removal of metal ions from liquid media.

### FIELD OF THE INVENTION

This invention relates to derivatised carbon, in particular to graphite and other forms of carbon having surfaces chemically modified to impart desired properties.

### BACKGROUND TO THE INVENTION

The accumulation and release of toxic substances into the environment, particularly toxic heavy metals, has increased significantly over the past few decades. The environmental impact of mining operations and heavy industry has led to the accumulation of high concentrations of toxic heavy metal ions such as Cu^{II}, Cd^{II}, Pb^{II} and Hg^{II} in lakes and rivers, these pollutants being largely nondegradable and recirculating in nature. The presence of heavy metals in aquatic media and drinking water are potentially dangerous to the health of both humans and aquatic life depending on the exposure levels and chemical form of the heavy metal. An example of the tragic human consequences of heavy metal pollution is the widespread poisoning of millions of people in countries such as Argentina, China, Mexico, Taiwan, India and in particular Bangladesh, where up to 60% of the Bangladeshi groundwater contains naturally occurring arsenic concentrations greatly in excess of the World Health Organisation's (WHO) guidelines of 10 ppb. As many salts of these heavy metal ions are water soluble, common physical methods of separation are rendered ineffective. There is a pressing need to develop a facile, rapid and inexpensive method of removing toxic heavy metal ions from aqueous media for use in drinking water filtration and/or environmental clean up.

Polypeptides such as poly-L-histidine, poly-L-aspartic acid, poly-L-glutamic acid and in particular poly-L-cysteine are known to chelate metal ions such as Cd^{II}, Pb^{II}, Ni^{II} and Cu^{II} and have been attached to various substrates and used in the trace analysis of these metals (Malachowski et al, Anal. Chim. Acta. 2003, 495, 151; Malachowski et al, Anal. Chim. Acta 2004, 517, 187; Malachowski et al, Pure Appl. Chem. 2004, 76, 777; Johnson et al, Anal. Chem. 2005, 77, 30; Howard et al, J. Anal. At. Spectrom. 1999, 14, 1209; and Jurbergs et al, Anal. Chem. 1997, 69, 1893). Biohomopolymers and other peptides possess significant advantages for metal extraction or reclamation over traditional techniques such as simple filtration or precipitation, as the latter are often unable to reduce the concentration of the target metals to meet strict environmental agency regulations.

Graphite surfaces can be chemically modified using a variety of relatively facile techniques such as physisorption and chemically or electrochemically initiated chemisorption of a given chemical or biological moiety. Graphite having derivatised surfaces may be used in a variety of applications, for instance as electrode materials in battery technology and as sensors. Although reactive groups such as hydroxyl and carboxyl moieties are known to be present on the surface of graphitic materials, the use of chemically derivatised graphite as a solid-state support for synthetic chemistry applications has been limited.

### SUMMARY OF THE INVENTION

The present invention provides carbon-based solid-state supports upon which to conduct synthetic, step-wise syntheses. This allows the derivatisation of the surface of such materials in a “building-block” fashion, to impart desired properties such as sensitivity to a target analyte. In this way, species such as amino acids, peptides, small proteins and nucleic acids can coupled to carbon (e.g. graphite) particles in a relatively facile manner. By varying the chemistry of the species that initially derivatises the carbon surface, various methods of coupling building-block molecules to the carbon surface are possible. In particular, the present invention provides derivatised carbon, especially graphite, to which is attached an amino acid or a derivative thereof. The amino acid may be monomer (e.g. cysteine) or a polypeptide (e.g. poly-L-cysteine), which is capable of binding metal ions. The invention is therefore particularly relevant to the detection and removal of toxic heavy metals from water and other liquid media.

According to a first aspect of the present invention, there is provided derivatised carbon in which an amino acid or a derivative thereof is attached to the carbon. The attachment may be direct or indirect, for example via a phenylamine group.

The present invention also provides a method of preparing a derivatised carbon in which the carbon is contacted with a nitrobenzenediazonium compound under conditions such that a nitrophenyl-derivatised carbon is produced.

The present invention also provides a method of preparing derivatised carbon in which the carbon is attached directly to the amino acid or derivative thereof via carboxyl groups on the surface of the carbon, the method comprising converting carboxyl groups on the surface of the carbon to acyl halide groups and then contacting the resultant product with the amino acid or derivative thereof.

The present invention also provides a carbon electrode comprising derivatised carbon of the invention.

The invention further provides an electrochemical device including an electrode of the invention. The electrochemical device may be in the form of an electrochemical sensor or reactor.

In addition, the present invention provides a method of removing metal ions from a liquid medium comprising contacting the medium with derivatised carbon of the invention.

Furthermore, the present invention provides a method of detecting the presence of metal ions in a liquid medium comprising subjecting the medium to voltammetric analysis using an electrochemical device of the invention.

Derivatised carbon of the invention may be useful in the detection, removal, sequestration and titration of metal ions from liquid media, including water and other aqueous media. Such metal ions include, for instance, Cd(II), Pb(II), Zn(II), Cu(II) and As(III) ions. The derivatised carbon may be in particulate form, for example in the form of a powder. Particulate materials such as graphite powder and glassy carbon powder are desirable because of their high surface area, which allows them to couple relatively large amounts of amino acids or derivatives thereof. Derivatised carbon of the invention may therefore be able to bind a significantly greater amount of metal ions than known modified solid-state materials.

### BRIEF DESCRIPTION OF THE DRAWINGS

FIG. 1 shows:

a) consecutive voltammograms showing the response of 4-nitrophenyl-derivatised carbon (“NPcarbon”) in pH 6.8 buffer;

b) overlaid voltammograms of blank graphite powder and aniline-derivatised carbon (“ANcarbon”) in acetonitrile containing 0.1 M tetrabutylammonium perchlorate (TBAP) as supporting electrolyte; and

c) consecutive voltammograms showing the response of 4-nitrobenzoic acid-derivatised carbon (“NBANcarbon”) in pH 6.8 buffer.

FIG. 2 shows:

a) the N₁ₛ region of the X-ray photoelectron spectroscopy (XPS) spectrum of ANcarbon; and

b) the N₁ₛ region of the XPS spectrum of NBANcarbon.

FIG. 3 shows:

a) the wide XPS spectrum of poly-S-benzyl-L-cysteine-derivatised carbon (“PSBCcarbon”) and

b) the wide XPS spectrum of poly-L-cysteine-derivatised carbon (“PCcarbon”).

FIG. 4 shows linear sweep stripping voltammograms for Cd²⁺ detection with standard additions of Cd²⁺. The inset shows the corresponding standard addition plot.

FIG. 5 shows the cadmium concentration profile remaining in a 10 cm³ sample of river water (original Cd(II) concentration ca. 1.5 mM) after exposure to 10 mg cysteine methylester-derivatised glassy carbon (“CysMeO-GC”).

FIG. 6 shows the cadmium concentration profile remaining in a 10 cm³ sample of mineral water (original Cd(II) concentration 50 ppb) after exposure to 10 mg CysMeO-GC.

FIG. 7 shows the copper concentration profile remaining in a 10 cm³ sample of river water after exposure to 10 mg CysMeO-GC for varying times.

FIG. 8 shows the concentration of As(III) remaining after exposure to 10 mg of PCcarbon powder, stirred for specified lengths of time. The curve shows a first order exponential decay fitted to the data.

FIG. 9 shows the concentration of As(III) remaining after exposure to 10 mg of CysMeO-GC powder, stirred for specified lengths of time. The curve shows a first order exponential decay fitted to the data.

FIG. 10 shows the concentration of As(III) remaining after exposure to 200 mg of CysMeO-GC powder to a 200 ppb As(II) solution, stirred for specified lengths of time. The curve shows a first order exponential decay fitted to the data.

FIG. 11 shows the concentration of As(III) remaining after exposure to 200 mg of CysMeO-GC powder to a 120 ppb As(III) solution in a Bangladeshi water sample, stirred for specified lengths of time. The curve shows a first order exponential decay fitted to the data.

FIG. 12 shows anodic stripping voltammograms of a 120 ppb As(III) Bangladeshi water sample exposed to 200 mg of CysMeO-GC spherical powder and stirred for 30 minutes. Linear sweep voltammetry (LSV) was performed at 100 mV/s, and standard additions of 2.4×10⁻⁷ M were used.

FIG. 13 shows an XPS spectrum of L-cysteine methyl ester-modified carbon powder (“CysOMe-carbon”).

FIG. 14 shows an baseline-corrected XPS spectrum of CysOMe-carbon powder after exposure to As^{III} showing the region of interest from 120 to 260 eV. The dotted lines show the Gaussian peak fitting performed using the MicroCal Origin software package.

FIG. 15 shows overlaid concentration-time profiles for the removal of Cd^{II} from a ca. 55 μM solution of Cd(NO₃)₂ in pH 5.0 acetate buffer comparing the efficacy of CysOMe-GC and CysOMe-carbon powder adsorbents.

FIG. 16 shows a concentration-time profile for the removal of trace amounts of As^{III} to below the WHO recommended limit of 10 ppb.

FIG. 17 shows overlaid Cd^{II} linear sweep anodic stripping voltammetry (LSASV) voltammograms with increasing 1 μM standard additions of Cd^{II} (0-20 μM). The inset shows the corresponding standard addition plot.

FIG. 18 shows overlaid As^{III} LSASV voltammograms with increasing 0.22 μM standard additions of As^{III} (0 to 2.2 μM). The inset shows the corresponding standard addition plot.

### DESCRIPTION OF VARIOUS EMBODIMENTS

The invention provides derivatised carbon to which is attached an amino acid or a derivative thereof. The amino acid or derivative may be attached directly or indirectly (i.e. via a linker) to the carbon. Of particular mention is carbon to which the amino acid or derivative is attached via a carboxyl or phenylamine group present on the carbon.

In one embodiment, the amino acid is a sulfur-containing amino acid, for instance, cysteine, glutathione, tyrosine or a derivative thereof. The sulphur-containing amino acid may have pendant thiol or thiol-like groups. The amino acid may be in the form of an ester, e.g. a methyl or ethyl ester, a particular example being L-cysteine methyl ester. Derivatives of amino acids include oligomers and polymers of amino acids. By way of example, a cysteine derivative may be polycysteine or cysteamine, while a glutathione derivative may be polyglutathione. An exemplary polymeric amino acid is an S-benzyl protected homopolymer containing 50 to 100 cysteine residues per polymer chain. The amino acid, or derivative thereof, may be protected or unprotected, an example being a polycysteine such as poly-S-benzyl-L-cysteine.

The carbon may be in particulate form, for example in the form of a powder. A particulate carbon may comprise particles having a diameter of between 1 and 100 μm, e.g. between 2 and 50 μm. Of particular mention are graphite powder, glassy carbon spherical powder and pyrolytic graphite forms. Alternatively, the carbon may be in the form of carbon nanotubes, for instance, multiwalled carbon nanotubes (MWCNTs).

Examples of derivatised carbons of the invention include glassy carbon modified with cysteine, glutathione or cysteamine or a derivative thereof, and a carbon powder modified with polycysteine or polyglutathione. It will be appreciated that the invention extends to other amino acid polymers and derivatives and also to monomers of amino acids and their thiol-containing derivatives, such as cysteine, coupled to glassy carbon. Particular examples include carbon powder (e.g. graphite powder or glassy carbon spherical powder) derivatised with cysteine or a derivative thereof (e.g. an ester of cysteine such as cysteine methyl ester, or a polymer of cysteine such as polycysteine or poly-S-benzyl-L-cysteine).

The derivatised carbon may be obtained by contacting carbon with a nitrobenzenediazonium compound under conditions such that a nitrophenyl-derivatised carbon is produced. The reaction may be carried out in the presence of a suitable reagent such as hypophosphorous acid. The nitrophenyl-derivatised carbon may be reduced to form an aniline-derivatised carbon. The product may be further reacted to produce a substituted aniline-derivatised carbon. In particular, the aniline-derivatised carbon may be reacted with an amino acid or derivative thereof (e.g. a polycysteine such as poly-S-benzyl-L-cysteine).

Derivatised carbon may also be obtained by converting carboxyl groups present on the surface of a carbon to acyl halide groups and then contacting the resulting product with an amino acid or derivative thereof. The acyl halide may be, for example, acyl chloride. Any carboxyl groups present on the amino acid or derivative thereof may be protected.

Derivatised carbon of the invention may be used in the detection (e.g. the electrochemical detection), titration or removal of metal ions from liquid media. The metal ions may be, for instance, one or more of Cd(II), Pb(II), Zn(II), Cu(II) and As(III) ions. The liquid medium may be, for instance, an aqueous medium.

Derivatised carbon of the invention, especially cysteine- or polycysteine-derivatised carbon, may be useful in the detection of arsenic. For example, the carbon may be provided in a relatively expensive drinking water filtration device. Conversely, to the extent that a derivatised carbon of the invention is selective for metal ions other than As(III), it may be incorporated into an arsenic sensor in order to remove ions such as Cu(II), which interfere in As(III) detection. Accordingly, the invention may provide inexpensive and attractive materials for use in water clean-up, the recovery or extraction of metals from effluents, and drinking water filtration, where natural supplies are often contaminated by toxic heavy metals such as arsenic and cadmium.

The invention further provides materials which may be useful in metal sequestration. By way of example, polycysteine anchored on carbon typically has a much higher metal uptake (per gram of material) than known substrates such as glass, polymer beads and the like. The density of sequestration units per surface area may also be much greater than for prior art substrates where nano-scale modification is used (e.g. in the case of nanotubes) is used, due to an increase in active surface area. Hence both the thermodynamics and the kinetic rate of metal ion uptake may be enhanced.

In particular, the present invention provides a solid-state support material in which the support is provided by coupling a biohomopolymer, in particular a polypeptide selected from poly-L-histidine, poly-L-aspartic acid, poly-L-glutamic acid and especially poly-L-cysteine, to graphite powder. As mentioned above, such polymers are known to chelate toxic heavy metals such as cadmium, lead, nickel and copper with very little affinity for alkali and alkaline earth metals such as sodium and calcium. A cysteine-, poly-L-cysteine-derivatised graphite powder of the invention may be used to quantitatively titrate metal ions, such as Cd(II) ions, from aqueous media. Due to the high surface area of graphite powder and the ability to couple large amounts of amino acid to it, cysteine- or polycysteine-modified carbon may chelate far greater amounts of Cd(II) ions than poly-L-cysteine attached to any other solid-state support material. Thus, derivatised carbon of the invention is particularly suited for use in toxic heavy metal recovery from industrial effluents, environmental cleanup and drinking water filtration.

The following Examples illustrate the invention.

### EXAMPLE 1

### Derivatisation of Graphite Powder with Poly-L-Cysteine

### Reagents and Chemicals

With the exception of potassium chloride (purchased from Riedel de Haën), all reagents were obtained from Aldrich and were of the highest grade available and used without further purification. The synthetic graphite powder used consisted of irregularly shaped particles of between 2 and 20 μm diameter and was purchased from Aldrich. All aqueous solutions were prepared using deionised water from an Elgastat UHQ grade system (Elga) with a resistivity of not less than 18.2 MΩ cm.

Solutions of known pH in the range pH 1.0 to pH 12.0 were prepared in deionised water as follows: pH 1.0, 0.10 M HCl; pH 1.7, 0.1 M potassium tetraoxalate; pH 4.6, 0.10 M acetic acid +0.10 M sodium acetate; pH 5.04, 0.5 M sodium acetate; pH 6.8, 0.025 M Na₂HPO₄+0.025 M KH₂PO₄; pH 9.2, 0.05 M disodium tetraborate; pH 10.5, 0.1 M disodium tetraborate; and pH 12.0, 0.01 M sodium hydroxide. These solutions contained in addition 0.10 M KCl as supporting electrolyte. pH measurements were performed using a Hanna pH213 pH meter.

### Instrumentation

Electrochemical measurements were recorded using a pautolab computer controlled potentiostat (Ecochemie) with a standard three-electrode configuration. Electrochemical experiments were carried out in a glass cell of volume 25 cm³. Either a basal plane pyrolytic graphite electrode (bppg, 5 mm diameter, Le Carbone) or boron doped diamond electrode (BDD, 3 mm diameter, Windsor Scientific Ltd.) electrode acted as the working electrode. A platinum coil (99.99%, Goodfellow) acted as the counter electrode. The cell assembly was completed using a saturated calomel electrode (SCE, Radiometer) as the reference electrode unless otherwise stated. All electrochemical experiments were carried out after degassing the solution using pure N₂ gas (BOC gases) for 30 minutes and were conducted at 20±2° C.

X-ray photoelectron spectroscopy (XPS) of the 4-nitrophenyl-derivatised carbon after reduction with Sn/HCl and after the coupling of 4-nitrobenzoic acid was performed on a Scienta ESCA300 instrument using X-ray radiation from the aluminium Ka band (hv=1486.7 eV), source setting 14 hv, 200 mA. All spectra were recorded using a pass energy of 150 eV and a take off angle of 90°. A slit width of 1.9 mm was used, unless otherwise stated. The base pressure in the analysis chamber was maintained at not more than 2.0×10⁻⁹ mbar.

XPS of the S-benzyl-protected poly-L-cysteine and the deprotected poly-L-cysteine was performed on a VG Clam 4 MCD analyzer system, using X-ray radiation from the Al K_{α} band (hv=1486.7 eV). All XPS experiments were recorded using an analyzer energy of 100 eV with a take-off angle of 90°. The base pressure in the analysis chamber was maintained at not more than 2.0×10⁻⁹ mbar. Each derivatised carbon sample studied was mounted on a stub using double sided adhesive tape and then placed in the ultra-high vacuum analysis chamber of the spectrometer. To prevent the sample from becoming positively charged when irradiated due to emission of photoelectrons, the sample surface was bombarded with an electron beam (10 eV) from a “flood gun” within the spectrometer's analysis chamber. Analysis of the resulting spectra was performed using Microcal Origin 6.0. Assignment of spectral peaks was determined using the UKSAF and NIST databases.

### General Reaction Scheme

Scheme I illustrates synthetic routes for derivatising graphite powder showing the principle behind the “building-block” chemistry and the coupling of poly-L-cysteine to graphite powder:

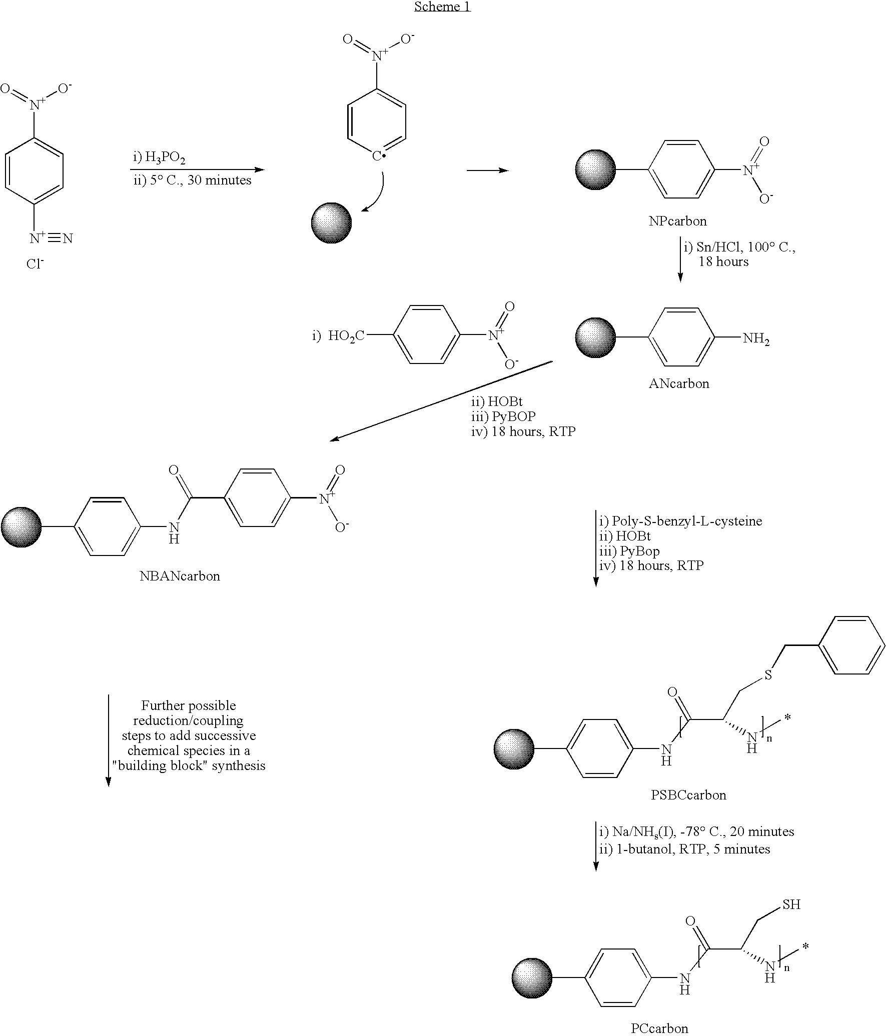

Derivatisation of Graphite Powder with 4-nitrophenyl to Form NPcarbon

First 0.5 g of graphite powder was stirred into 10 cm³ of a 5 mM solution of Fast Red GG (4-nitrobenzenediazonium tetrafluoroborate), to which 50 cm³ of hypophosphorous acid (H₃PO₂, 50% wlw in water) was added. Next, the solution was allowed to stand at 5° C. for 30 minutes with gentle stirring, after which the solution was filtered by water suction and washed with deionised water to remove any excess acid and finally with acetonitrile to remove any unreacted diazonium salt. The 4-nitrophenyl-derivatised graphite powder (“NPcarbon”) was then air-dried by placing inside a fume hood for a period of 12 hours after which they were stored in an airtight container prior to use (Pandurangappa et al, Analyst, 2002, 127 1568; and Pandurangappa et al, Analyst, 2003, 128, 473).

### Reduction of NPcarbon to Form ANcarbon

NPcarbon powder (1.02 g) and tin (1.63 g, 13.7 mmol) were suspended in water (12 mL). Concentrated hydrochloric acid (4.5 ml, 53.8 mmol) was added and the mixture heated to reflux. The reaction mixture was stirred at 100° C. under an atmosphere of argon. After 18 h the mixture was filtered and the solid washed with hydrochloric acid (100 mL of a 1M aqueous solution), methanol (100 mL), potassium hydroxide (50 mL of a 1M aqueous solution) and methanol (50 mL). The solid was dried in vacuo to afford a black powder (180.4 mg) of the reduced form of NPcarbon consisting of p-aniline moieties covalently derivatised to the graphite surface (“ANcarbon”).

Coupling of 4-nitrobenzoic Acid to ANcarbon

ANcarbon (500 mg), 1-hydroxybenzotriazole hydrate (HOBt, 670 mg, 5.0 mmol), benzotriazol-1-yl-oxytripyrrolidinophosphonium hexafluorophosphate (PyBop, 2.6 g, 5 mmol) and p-nitrobenzoic acid (840 mg, 5 mmol) were placed in a flask and DMF (8 mL) added. Ethyl diisopropylamine (1.7 mL, 10 mmol) was added. The reaction mixture was stirred under argon at room temperature. After 18 h the mixture was filtered and the solid washed with methanol (50 mL), acetonitrile (50 mL) and DCM (50 mL). The solid was dried in vacuo to afford a black powder consisting of 4-nitrobenzoic acid coupled to the ANcarbon surface via an amide linkage (“NBANcarbon”).

### Voltammetric and XPS Characterisation of NPcarbon, ANcarbon and NBANcarbon

Voltammetric characterisation of the derivatised NPcarbon, ANcarbon and NBANcarbon was carried out over the range pH 1.0 to pH 12.0, after first separately abrasively immobilising each derivatised carbon onto the surface of a bppg electrode as described in Leventis et al, Talanta, 2004, 63, 1039.

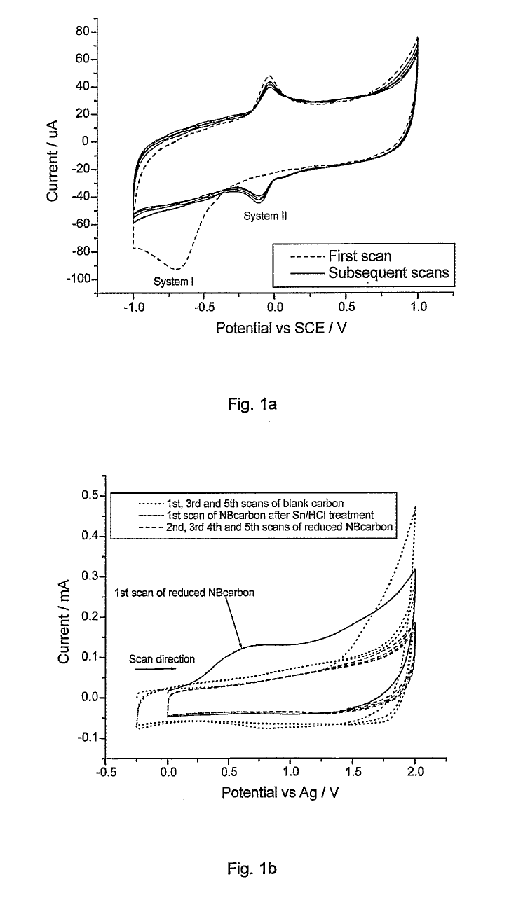

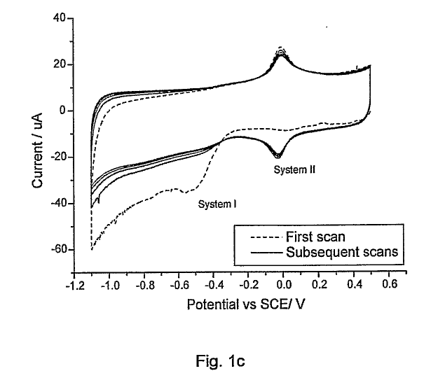

FIG. 1a shows the voltammetry of NPcarbon at pH 6.8. Upon first scanning in a reductive direction a large reduction wave was observed at ca. −0.685 V vs. SCE labelled as “System I” in FIG. 1a. When the scan direction was reversed at −1.0 V vs. SCE, no corresponding oxidation wave for System I was observed, indicating that the process was electrochemically irreversible. However, an oxidation wave was observed at ca. +0.025 V vs. SCE. On subsequent scans the corresponding reduction wave is observed at ca. −0.095 V vs. SCE corresponding to an electrochemically almost-reversible process, termed “System II”. The electrochemically irreversible System I is not present in subsequent scans indicating that all the 4-nitrophenyl moieties have been reduced.

The observed voltammetric behaviour and their wave-shapes are consistent with previous studies of NPcarbon (Pandurangappa et al, Analyst, 2002, 127, 1568) and corresponds to the electrochemical reduction of the surface-bound nitro groups in aqueous media. Scheme 2 illustrates this behaviour for the generic example of nitrobenzene itself (Pandurangappa et al, Analyst, 2002, 127, 1568; and Rubinstein, J. Electroanal. Chem., 1971, 29, 309):

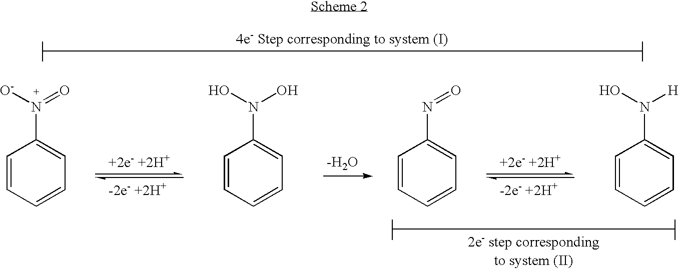

In this mechanism, System I corresponds to the chemically and electrochemically irreversible reduction of the nitro group in a four-electron, four-proton process to form the arylhydroxylamine. This then undergoes an electrochemically almost-reversible two-electron, two-proton oxidation (System II) to form the arylnitroso species. This voltammetric behaviour was observed at every pH studied, although, due to concomitant proton transfer, the peak potentials for both Systems I and II depended on pH and vary by 55.4 and 54.4 mV/pH unit respectively in a linear, Nernstian fashion over the range pH 1.0 to pH 12.0 in agreement with previous studies.

A well established voltammetric characterisation protocol (Leventis et al, Talanta 2004, 63, 1039; and Wildgoose et al, Talanta, 2003, 60, 887), was then carried out over the almost-reversible System II at each pH studied and confirmed that the 4-nitrophenyl moieties were indeed confined to the surface of the graphite particles.

Voltammetric characterisation of ANcarbon revealed that no voltammetric waves corresponding to either System I or II were observed. Thus it could be concluded that all the 4-nitrophenyl groups were reduced to the corresponding aniline-like moieties. Voltammetry of ANcarbon in acetonitrile containing 0.1 M tetrabutylammonium perchlorate (TBAP) showed, in the first scan, an oxidative wave at ca. +0.700 V vs. a silver pseudo-reference electrode at potentials corresponding to the one-electron oxidation of aniline to its radical cation (FIG. 1b).

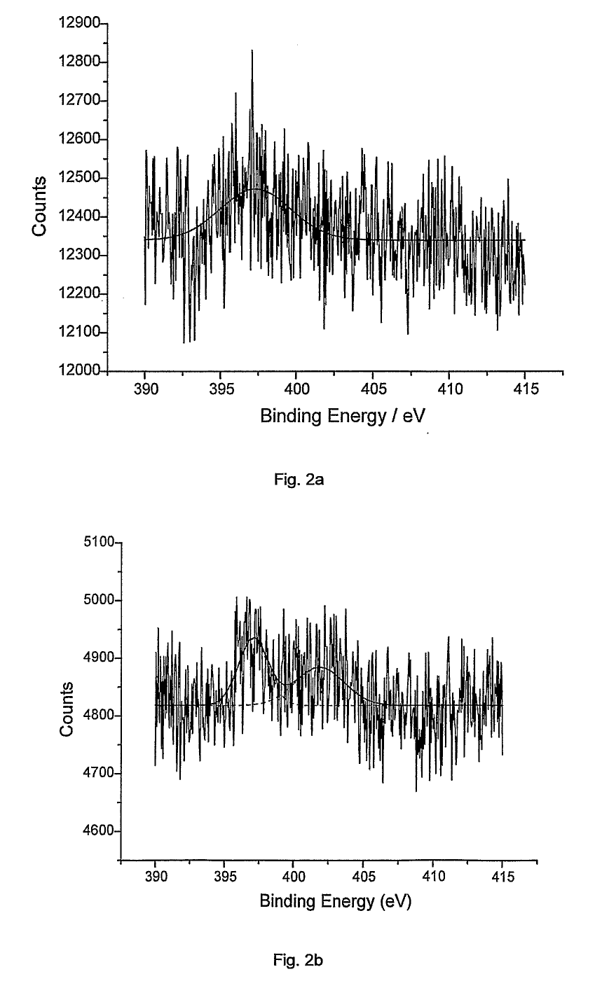

The ANcarbon was further characterised using XPS. FIG. 2a shows that a single peak is observed in the N₁ₛ region of the spectrum with a binding energy of 400.1 eV consistent with an aromatic amine moiety. No signals at binding energies corresponding to photoelectrons emitted from the N₁ₛ or O₁ₛ levels within a nitro moiety were observed.

Voltammetric characterisation of the NBANcarbon revealed that the expected characteristic reduction of the nitro group is once again observed and that the voltammetry corresponds to a surface bound species (FIG. 1c). FIG. 2b shows the N₁ₛ region of the XPS spectrum of NBANcarbon. Two peaks are observed with binding energies of 400.6 eV and 405.4 eV and an almost 1:1 ratio of peak heights. Comparison with XPS databases confirms that these peaks correspond to nitrogen atoms in the amide and nitro groups respectively. Furthermore, Gaussian deconvolution of the O₁ₛ region of the spectrum (not shown) reveals peaks with binding energies of 530.7 eV and 533.6 eV consistent with oxygen atoms within an amide and an aromatic nitro group respectively. In light of these results, it can be concluded that coupling takes place solely between the 4-nitrobenzoic acid molecules and the aniline-like moieties on the surface of ANcarbon.

### Coupling of Poly-S-benzyl-L-cysteine to ANcarbon to Form PSBCcarbon

Poly-S-benzyl-L-cysteine (PSBC, 170 mg, 0.02 mmol) was dissolved in 1,4-dioxane (3 ml). Trimethylsilyl chloride (5.6 μL, 0.04 mmol) in DMF (3 mL) was added to increase the solubility of the peptide homopolymer. The reaction mixture was stirred under argon at 50° C. After 1 h the reaction mixture was cooled to room temperature. Ethyl diisopropylamine (6.5 μL, 0.04 mmol) was added and the mixture cooled to 0° C. before addition of 9-fluoroenylmethoxycarboxyl chloride (Fmoc, 5.7 mg, 0.02 mmol). The mixture was allowed to warm to room temperature. After 1 h 30 min the solvent was removed in vacuo to afford a white solid. To the residue was added 1-hydroxybenzotriazole hydrate (HOBt, 4.2 mg, 0.2 mmol), benzotriazol-1-yl-oxytripyrrolidinophosphonium hexafluorophosphate (PyBop, 11.6 g, 0.02 mmol), ANcarbon (104 mg) and DMF (10 mL). Ethyl diisopropylamine (17.7 μL, 0.04 mmol) was added. The reaction mixture was stirred under argon at room temperature. After 19 h the mixture was filtered and the solid washed with DMF (10 mL), methanol (10 mL), acetonitrile (50 mL) and DCM (50 mL). The solid was dried in vacuo to afford a black powder (200 mg) consisting of S-benzyl protected poly-L-cysteine coupled to ANcarbon via an amide linkage (“PSBCcarbon”).

### Deprotection of PSBCcarbon

Deprotection of the thiol groups in the poly-L-cysteine was achieved using a Birch reduction process. Liquid ammonia (ca. 10 mL) was condensed into a flask containing PSBCcarbon (124 mg) and sodium (120 mg, 5.2 mmol). The solution was stirred under argon at −78° C. After 20 min 1-butanol (0.3 mL) was added and the reaction stirred for a further 5 min before being allowed to warm to room temperature. Once the ammonia had evaporated ammonium chloride (ca. 4 mL of a saturated aqueous solution) was added to quench the reaction. The suspension was filtered and the solid washed with water (20 mL), methanol (20 mL) and DCM (20 mL). The solid was dried in vacuo to afford a black powder (106 mg) consisting of poly-L-cysteine coupled to ANcarbon via an amide linkage (“PCcarbon”).

### XPS Characterisation of PSBCcarbon and PCcarbon

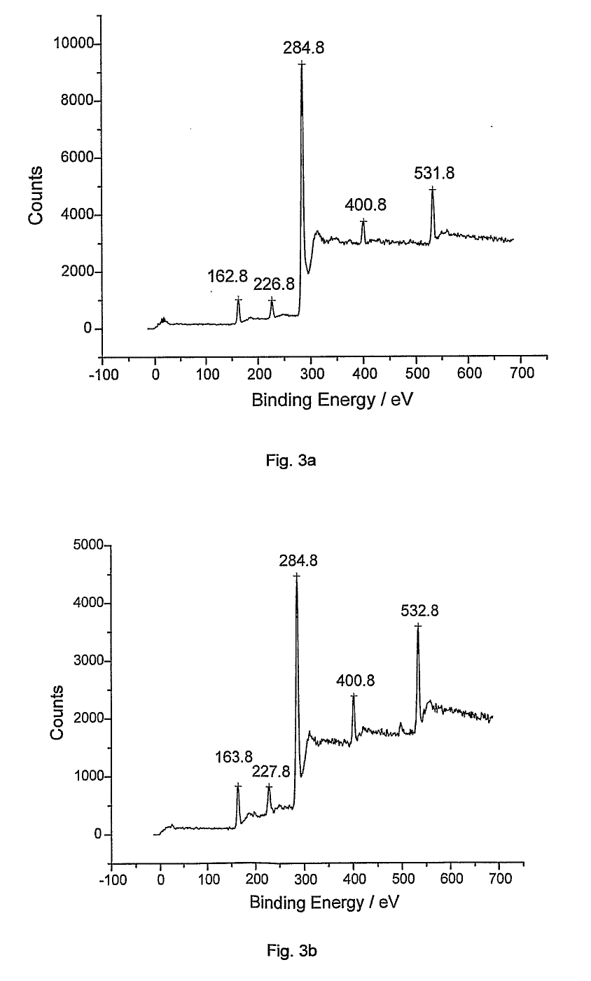

FIGS. 3a and 3b show the resulting XPS spectra for PSBCcarbon and PCcarbon respectively. Two peaks with binding energies of 162.5 eV and 226.5 eV corresponding to photoelectrons emitted from the S_{2p3\2} and the S₂ₛ levels were observed in the PSBCcarbon in excellent agreement with literature values for S-benzyl protected polycysteine. In the deprotected PCcarbon the binding energies of the S_{2p3/2} and the S₂ₛ photoelectrons were shifted slightly to 163.5 eV and 227.5 eV, again in excellent agreement with literature values for the free thiol in polycysteine. For both the PSBCcarbon and the PCcarbon the O₁ₛ and N₁ₛ peaks are located at 531.5 eV and 400.5 eV respectively are dominated by the contribution from the amide linkages in the polycysteine and are in excellent agreement with literature values. Elemental analysis of both the PSBCcarbon and PCcarbon samples revealed that the relative amounts of poly-L-cysteine coupled to the graphite surface did not change after deprotection of the thiol groups using a Birch reduction with the total sulphur oxygen and nitrogen signals accounting for ca. 7±1.4% each of the surface elemental composition, indicating that a relatively large amount of polycysteine was coupled to the surface. Thus it can be concluded that poly-L-cysteine coupled to ANcarbon and remained coupled after carrying out a Birch reduction to deprotect the thiol groups within the poly-L-cysteine.

### EXAMPLE 2

### Quantitative Analysis of Cadmium in Aqueous Media Using PCcarbon

The uptake of Cd²⁺ from aqueous solutions was monitored electrochemically using a linear-sweep stripping voltammetric (LSV) stripping protocol at a boron doped diamond (BDD) electrode developed by Banks et al, Talanta, 2004, 62, 279).

The optimised pH for Cd²⁺ detection is pH 5 and therefore a 0.05M sodium acetate buffer (pH 5.04) was used for both the chelation of Cd²⁺ by the PCcarbon and the LSV detection of the amount of Cd²⁺ chelated. The LSV protocol for cadmium detection involved depositing the Cd²⁺ on the BDD electrode as Cd⁰ by holding the potential at −1.5 V vs. SCE for 60 s whilst stirring the solution. LSV was then carried out by scanning the potential from −1.1 V to −0.3 V at 100 mVs⁻¹ and a cadmium stripping peak observed at ca. −0.8 V vs. SCE. To verify the accuracy of this protocol, a “blind” solution of Cd(NO₃)₂ was analysed by standard additions of 5 nM Cd²⁺ and a standard addition plot of peak height vs. Cd²⁺ concentration constructed.

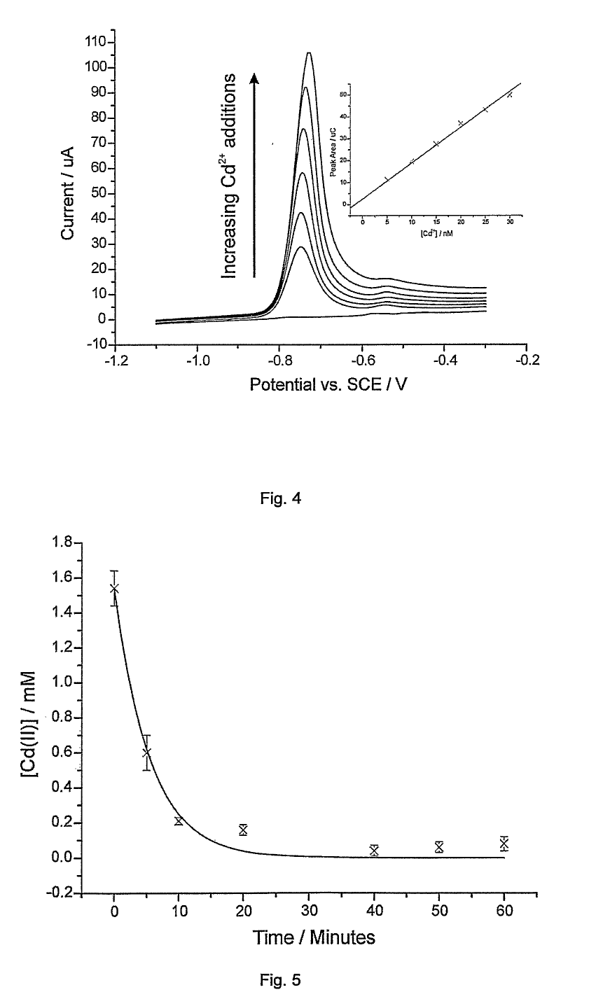

FIG. 4 shows the overlaid resulting LSV voltammograms for increasing amounts of Cd²⁺ and the resulting standard addition plot (inset). The Cd²⁺ concentration was determined by the LSV protocol to be 20.5 nM±0.1 nM with a limit of detection (3σ) of 0.2 nM. The actual Cd²⁺ concentration was 20 nM±0.1 nM demonstrating that the LSV protocol was an accurate method for trace Cd²⁺ determination over the concentration range 1-100 nM.

In order to measure the amount of Cd²⁺ chelated by PCcarbon a 1 mM Cd(NO₃)₂ solution was made up in pH 5 sodium acetate buffer. A 10 μL sample of this was then removed and diluted by a factor of 10⁵ in order for the initial Cd²⁺ concentration to be measured by the LSV protocol. Next 5 mg, 10 mg and 20 mg of PCcarbon were added to 10 cm³ of the 1 mM, 2 mM and 3 mM Cd(NO₃)₂ respectively and stirred for ten minutes. The PCcarbon was then filtered off and again a 10 μL sample of the filtrate was removed and diluted before the amount of Cd²⁺ remaining in the sample was measured using the LSV protocol. This procedure was repeated three times for each amount of PCcarbon added

Table 1 shows the amount of Cd²⁺ chelated for varying masses of PCcarbon. The experiments were repeated with the length of time the PCcarbon was stirred with Cd²⁺ varied from ten minutes to 12 hours. Increasing the exposure time of Cd²⁺ to PCcarbon was not found to increase the amount of Cd²⁺ chelated. A similar experiment was carried out with blank graphite powder for comparison. The uptake of Cd²⁺ by blank graphite powder was not measurable. From the results presented in Table 1 it was possible to calculate that PCcarbon chelates 1218 μmol±200 μmol of Cd²⁺ per gram of PCcarbon.

TABLE 1

| | | | [Cd²⁺] | Mass of Cd²⁺ |
|---|---|---|---|---|
| Mass of | Initial [Cd²⁺] | Final [Cd²⁺] | chelated by | chelated |
| PCcarbon/ | determined | determined | PCcarbon/ | per mg of |
| mg | by LSV/mM | by LSV/mM | mM | PCcarbon/mg |
| 5 | 1.1 | 0.5 | 0.6 | 0.14 |
| 10 | 2.0 | 0.6 | 1.4 | 0.16 |
| 20 | 3.1 | 0.5 | 2.6 | 0.14 |

The amount of Cd²⁺ chelated by varying masses of PCcarbon exposed to 10 cm³ solutions of varying Cd²⁺ for 10 minutes.

The uptake of Cd²⁺ by PCcarbon was shown to be up to one hundred times greater per gram than previous studies where polycysteine was coupled to other substrates (Jurbergs et al, Anal. Chem., 1997, 69, 1893; Malachowski et al, Pure Appl. Chem., 2004, 76, 777; Johnson et al. Anal. Chem. 2005, 77,30; and Howard et al, J. Anal. At. Spectrom., 1999, 14, 1209). Without wishing to be bound by theory, it is believed that this may be due to the large surface area of graphite powder and the large proportion of 4-nitrophenyl groups that can be coupled to the numerous edge-plane-like defect sites on the carbon surface, allowing a far greater amount of polycysteine to be coupled to graphite powder than to other solid-state supports. Furthermore, the quantitative titration of Cd²⁺ ions by PCcarbon occurs rapidly (<10 minutes) upon exposure of the PCcarbon to the cadmium (II) solution.

Previous studies have demonstrated that Cd²⁺ can be quantitatively recovered from polycysteine using nitric acid as a result of tertiary conformational changes, rather than simple proton exchange with the thiol groups (Howard et al, J. Anal. At. Spectrom., 1999, 14, 1209; and Miller et al, Anal. Chem., 2001, 73, 4087). Cadmium ions were recovered from the PCcarbon by stirring the filtered PCcarbon samples in 1M HNO₃. After stirring each sample of PCcarbon in 10 cm³ 1.0 M HNO₃ for either 30 minutes or 5 hours, the suspension was filtered. A 10 μL sample of the filtrate was removed, and diluted in pH 5 buffer before the amount of Cd²⁺ remaining in the sample was measured using the LSV protocol. In each instance, irrespective of whether the sample was treated for 30 minutes or 5 hours, 40%±10% of the chelated Cd²⁺ was recovered. This is in agreement with the studies of Howard et al, who found that polycysteine exhibits both weak and strong binding sites for Cd²⁺.

### EXAMPLE 3

### Derivatisation of Graphite Powder and MWCNTs with Tyrosine

4-Nitrophenyl groups were coupled to graphite and MWCNTs via the diazonium salt chemistry described in Example 1. The nitro group was reduced with Sn/HCl to produce aniline-modified carbon and MWCNTs. The aniline group was then diazotised and coupled to tyrosine to produce a material capable of metal chelation and also a route for further coupling amino acid- or thiol-containing molecules to the tyrosine-modified carbon and MWCNTs. The amine groups of the aniline moieties on the surface of the derivatised carbon and MWCNTs were also converted to thiol groups, for use in metal chelation/recovery.

### EXAMPLE 4

### Derivatisation of Glassy Carbon Powder with L-cysteine Methyl Ester

2 g Glassy carbon spherical powder (GC, 10-20 μm diameter, Type I, Alfa Aesar) was stirred with 10 cm³ SOCl₂ for 1 hour after which it was washed with dry CH₃Cl. This converts the carboxyl surface groups to the acyl-chloride analogues. This material was then reacted with 0.5 g of L-cysteine-methylester hydrochloride salt (Sigma-Aldrich) in 10 cm³ dry CH₂Cl₂, with stirring and the slow addition of 0.27 cm³ Et₃N. The reaction mixture was then stirred for 12 hours (overnight) to produce L-cysteine methylester-derivatised GC spherical powder (“CysMeO-GC”). This process is illustrated in Scheme 3:

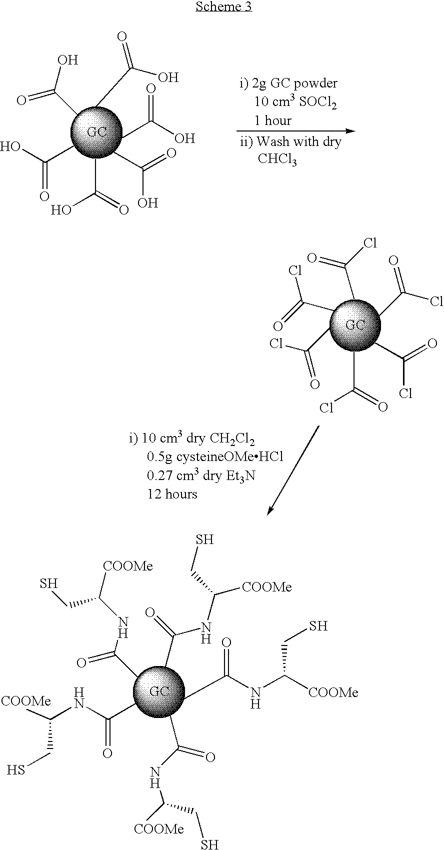

In a similar procedure, glassy carbon spherical powder was coupled with glutathione (reduced form, <99%, Aldrich) and cysteamine hydrochloride salt (Acros Organics).

### EXAMPLE 5

### Removal of Cadmium from Water Using CysMeO-GC Powder

### Detection of Cadmium

The linear sweep voltammetry (LSV) stripping protocol used was based on a previous detection protocol (Kruusman et al, Electroanalysis, 2004, 16, 399). A boron doped diamond electrode (BDD, diameter of 3 mm, Windsor Scientific) was used as the working electrode, with a platinum coil and saturated calomel electrode (SCE, Radiometer) acting as counter and reference electrodes respectively. The electrochemical experiments were carried out using a computer controlled potentiostat (μAutolab) in pH 5.04 0.05M sodium acetate buffer with 0.1 M KCl added as supporting electrolyte.

LSV detection of Cd(II) was carried out using the following parameters: a 10 μL aliquot of the sample to be tested was added to 10 cm³ of the sodium acetate buffer. Cadmium was deposited onto the BDD electrode at a potential of −1.5 V vs. SCE, for 60 s with stirring. The potential was then swept at 100 mVs⁻¹ from −1.1 V to −0.6 V vs. SCE with a cadmium stripping peak observed at ca. −0.780 V vs. SCE. Standard additions of 0.1 μM Cd(II) were then added over the range 0.1-1.0 μM and a corresponding addition plot was constructed and used to calculate the background Cd(II) concentration in the original sample.

Removal of Cadmium from River Water

A sample of river water was taken (untreated) from the River Cherwell in Oxford. A 10 cm³ sample of this river water was spiked to produce a cadmium(II) concentration of ca. 1.5 mM to simulate an environmentally disastrous spillage of toxic cadmium waste. This connection is the calculated average Cd(II) concentration in the River Neva which flows through St Petersburg, Russia and which is well known to be heavily polluted. 10 mg of CysMeO-GC powder was then added to this “real” matrix sample and stirred. The sample was filtered and a 10 μL aliquot removed for analysis using the LSV Cd(II) stripping protocol given above every after 5 minutes and then at every 10 minute interval for 1 hour.

FIG. 5 shows the resulting Cd(II) concentration profile. It is apparent that ca. 87% of the Cd(II) was removed from the sample by 10 mg of CysMeO-GC powder. The residual Cd(II) concentration was approximately half that of the calculated drinking water concentration of Cd(II) in the St Petersburg water supply out of the tap, which is still above the WHO, EU and EPA guidelines. CysMeO-GC powder may be used as a cheap and highly effective material for use in environmental clean up and/or metal ion sequestration.

Removal of Cadmium from Mineral Water

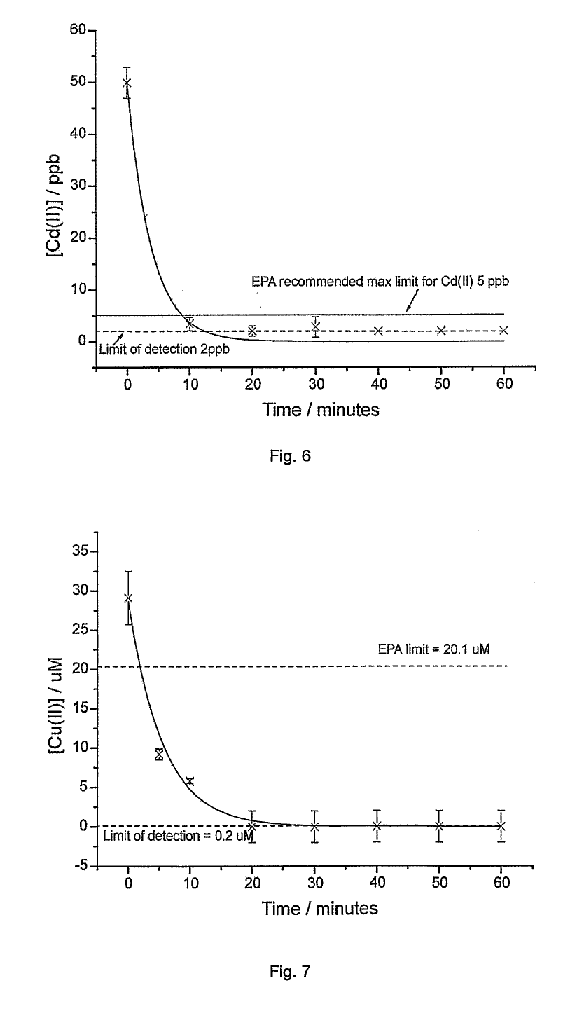

The contamination of drinking water supplies was simulated by spiking a 10 cm³ sample of Evian mineral water Cd(II) to produce a Cd(II) concentration of 50 ppb (parts per billion), which is ten times the EPA recommended maximum limit for drinking water. This “real” matrix was then stirred with 10 mg CysMeO-GC powder and analysed as described above. The resulting removal of Cd(II) is shown in FIG. 6.

Within ten minutes of exposure to CysMeO-GC powder the Cd(II) concentration in the mineral water was below the EPA recommended maximum limit of 5 ppb. Cys-GC is therefore an excellent material for use in drinking water filtration to remove toxic heavy metals such as Cd(II).

### EXAMPLE 6

### Removal of Copper from Water Using CysMeO-GC Powder

### Detection of Copper

The square wave voltammetry (SWV) stripping protocol used was based on a previous detection protocol (Banks et al, Phys. Chem. Chem. Phys., 2003, 5, 1652). A 50 μm diameter gold disc electrode (<99.99%, Goodfellow) was used as the working electrode, with a platinum coil and saturated calomel electrode (SCE, Radiometer) acting as counter and reference electrodes respectively. The electrochemical experiments were carried out using a computer controlled potentiostat (μAutolab) in pH 2.00 0.1 M phosphoric acid (H₃PO₄) buffer with 0.1 M KCl added as supporting electrolyte.

SWV detection of Cu(II) was carried out using the following parameters: frequency 50 Hz, step potential 2 mV, amplitude 25 mV. A 0.5 cm³ aliquot of the sample to be tested was added to 9.5 cm³ of the phosphoric acid buffer. Copper was deposited onto the working electrode at a potential of −1.5 V vs. SCE, for 15 s with stirring. The potential was then swept −1.0 V to +0.6 V vs. SCE with a copper stripping peak observed at ca. −0.05 V vs. SCE. Standard additions of 1.0 μM Cu(II) were then added over the range 1.0-10.0 μM and a corresponding addition plot was constructed and used to calculate the background Cu(II) concentration in the original sample.

Removal of Copper from River Water

A 10 cm³ sample of River Cherwell water (untreated) was analysed using the SWV copper stripping protocol outlined above and found to have a Cu(II) concentration of ca. 30 μM which is just above the EPA limit fo 1.3 mg L⁻¹ or 20.1 μM and was therefore used without spiking the Cu(II) concentration. Again the sample was exposed to 10 mg of CysMeO-GC and analysed at various intervals for one hour to measure the remaining Cu(II) concentration. FIG. 7 shows the resulting removal of Cu(II) from the sample.

### EXAMPLE 7

### Removal of Arsenic from Water Using PCcarbon and CysMeO-GC Powder

### Reagents and Chemicals

All chemicals used were of analytical grade and were used as received without any further purification. These were: sodium (meta) arsenite (Fluka, +99.0%) and nitric acid (Aldrich, 70%, double distilled PPB/Teflon grade with trace metal impurities in parts per trillion determined by ICP-MS). All solutions were prepared with deionised water of resistivity not less than 18.2 MΩ cm (Vivendi water systems). A sample of drinking water was obtained from Bangladesh.

### Instrumentation

Voltammetric measurements were carried out using a μ-Autolab III (ECO-Chemie) potentiostat. All measurements were conducted using a three electrode cell. The working electrode was a gold micro disk electrode (1 mm diameter), which was constructed in house by sealing a gold wire into Teflon housing. The counter electrode was a bright platinum wire, with a saturated calomel electrode (Radiometer) as the reference. The gold electrode was polished using a 0.1 μm alumina slurry on a soft lapping pad.

An ultrasonic horn, model CV 26 (Sonics and Materials Inc.) operating at a frequency of 20 kHz fitted with a 3 mm diameter titanium alloy microtip (Jencons) was used for sonovoltammetric studies. The intensity of the ultrasound was determined calorimetrically (Banks et al, Phys. Chem. Chem. Phys. 2004, 6, 3147; Magulis et al, Russ. J Phys. Chem. 1969, 43, 592; and Magulis et al, Ultrasonic. Sonochem., 2003, 10, 343) and was found to be 57 Wcm⁻² at 10%. The working electrode was placed in a face-on arrangement to the ultrasonic horn and the horn was immersed beyond the shoulder of the stepped tip to ensure that ultrasound was efficiently applied to the solution. For arsenic detection the voltammetric curves were baseline corrected using autolab software, which utilises a third-order polynomial correction.

### Removal of Arsenic Using PCcarbon

Polycysteine-derivatised carbon powder was tested for its ability to complex As(III) in pure water. As(III) concentrations were determined using anodic stripping voltammetry (ASV) at a gold electrode assisted by ultrasound during the deposition process. Power ultrasound to significantly enhance the sensitivity of arsenic detection using ASV at a gold electrode. The optimised conditions reported in Simm et al, Electroanalysis 2005, 17, 335 were used. A control experiment was performed before each sample was exposed to the complexing ligands to ensure the concentration of As(III) determined by the standard additions method was correct to within the detection limits of the procedure.

A 1.1 mM solution of As(III) was prepared from sodium (meta) arsenite dissolved in ultra pure water at pH 5.4, 25 mL of the solution was placed in a stirred flask to which 10 mg of the polycysteine carbon powder (PCcarbon) was added. At intervals of 10,30 and 60 minutes, a 50 μL sample was taken from the solution, which was then diluted down into 0.1 M nitric acid to trace levels for analysis. The analysis was performed by holding the gold electrode at −0.6 V (vs. SCE) for 60 s, ultrasound was used during this period at a horn to tip distance of 20 mm and an amplitude of 5%. The potential was then swept positively to 1 V (vs. SCE) from the deposition potential at a scan rate of 100 mV/s, revealing an arsenic stripping signal at ˜0.1 V (vs. SCE). For each analysis this initial value was measured 3 times and an average value calculated. Additions of 2.4×10⁻⁷ M As(III) were then performed each measurement which was repeated three times in order to determine the original concentration of As(III) present by the standard addition method.

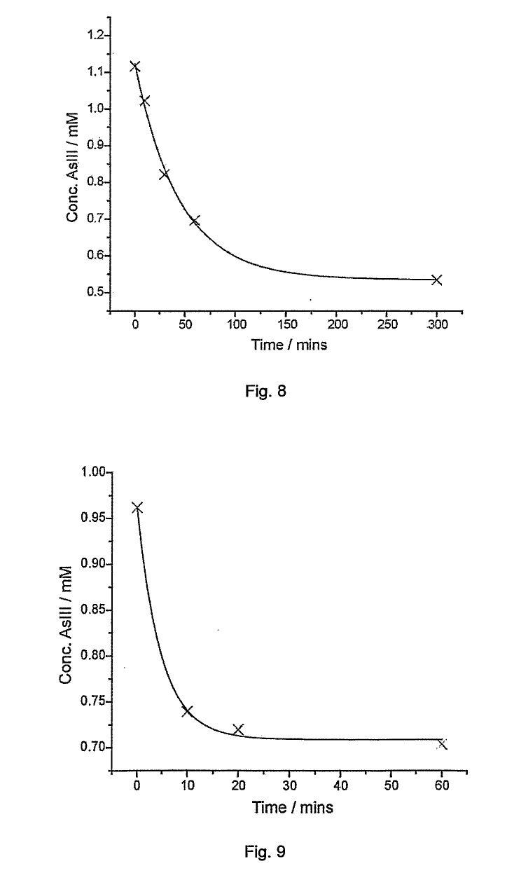

FIG. 8 shows the reduction in As(III) concentration over time, after 60 minutes of stirring the concentration of As(III) has dropped from 1.1 mM to 0.7 mM a 36% decrease, a first order exponential decay line has been fitted through the points. The solution was then left for a period of 20 days without further stirring after this time the concentration was found to have dropped to 0.55 mM.

Removal of Arsenic Using CysMeO-GC powder

A 0.98 mM solution of As (III) was prepared from sodium (meta) arsenite dissolved in ultra pure water at pH 5.4, 25 mL of the solution was placed in a stirred flask to which 10 mg of the Cys-GC powder was added. At intervals of 10, 20 and 60 minutes, a 50 μL sample was taken from the solution which was then diluted down in 0.1 M nitric acid to trace levels for analysis.

FIG. 9 shows the reduction in As(III) concentration over time, after 60 minutes of stirring the concentration of As(III) has dropped from 0.98 mM to 0.7 mM a 28.6% decrease. The solution was then left 3 days without further stirring however no further decrease in arsenic concentration was found after this time.

Experiments were then carried out at trace levels such that would be expected to be found in drinking water from areas such as Bangladesh (Anawar et al, Environment International 2002, 27, 597). A sample was prepared to an As(III) level of 200 ppb (2.66 μM) 4 times greater than the Bangladeshi limit of 50 ppb. 200 mg of CysMeO-GC powder was then placed in 25 mL of the sample which was then stirred for a specified length of time before filtration of the CysMeO-GC powder using filter paper in order to stop the complexation of As(III) by cystiene. The sample was then diluted 1:1 into a 0.1 M nitric acid solution for analysis.

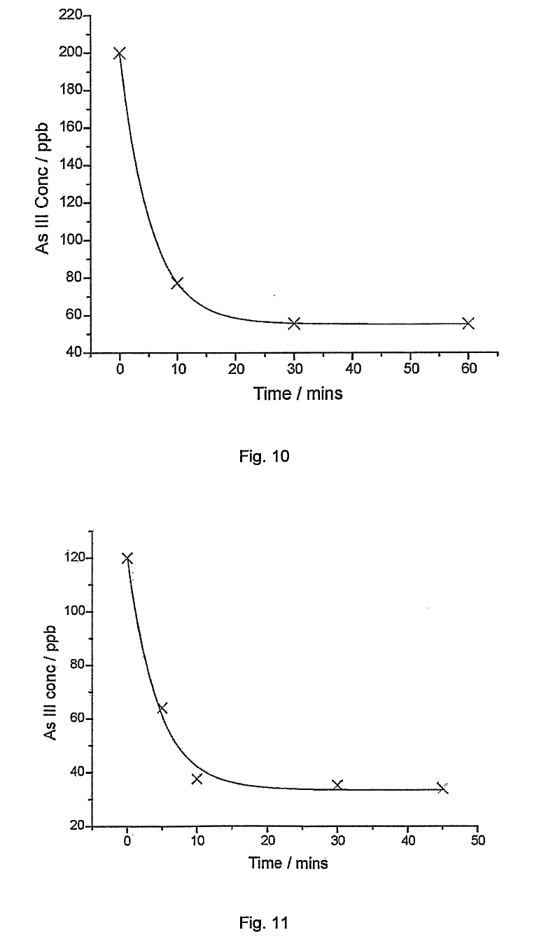

FIG. 10 shows that after only ten minutes the arsenic concentration has been significantly reduced from 200 ppb to 77 ppb, and after 30 minutes the level has dropped to 55 ppb. Analysis at 60 minutes shows that the concentration of arsenic has remained constant at this level (a 73% decrease) leaving the concentration of As(III) present just above the Bangladeshi safe drinking limit.

A real sample was then used to test the ability of the CysMeO-GC powder to complex arsenic in an authentic Bangladeshi well water sample. The sample was first tested by the ASV method to determine the concentration of As(III) present. However, the concentration of As(III) was found to be below the detectable limit (1×10⁻⁸ M), and so the water sample was spiked to a value of 120 ppb for use in the experiments. As in the experiments described above, 200 mg of the CysMeO-GC powder was added to 25 mL of the water sample which was then stirred for a specified time (5, 10, 30 and 45 minutes), before being filtrated to remove the powder from the solutions. Once again the sample was diluted 1:1 into 0.1 M nitric acid for the analysis experiments.

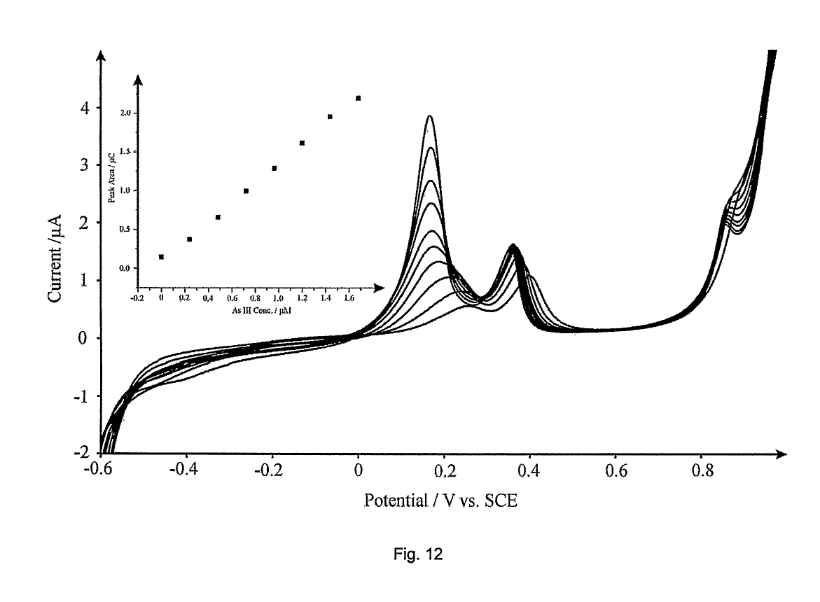

FIG. 11 shows the results of the analysis fitted to a first order exponential decay. After only 5 minutes of stirring the concentration of arsenic present had dropped by 47% to 64 ppb, at 10 minutes the concentration is found to have dropped further by 69% to 38 ppb (i.e. 12 ppb below the Bangladeshi safe drinking limit). After 45 minutes, the drop in concentration has levelled off at 34 ppb, or 28% of the original value. As the analysis was conducted in a real sample rather than pure water the experiment was exposed to many trace metals generally found in Bangladeshi water supplies (copper, lead, mercury etc; Anawar et al, Environment International 2002, 27, 597). FIG. 12 shows the ASV plots from the analysis of the 30 minute sample, a large stripping wave can be seen at approximately 0.4 V vs. SCE, due to one of these contaminants.

### EXAMPLE 8

### Derivatisation of Carbon Powder with L-cysteine Methyl Ester

### Reagents and Equipment

All reagents were purchased from Aldrich, with the exception of the glassy carbon microspherical powder (Alfa Aesar, Type I, diameter 10-20 μm) and potassium chloride (Reidel de Haen) and were of the highest commercially available grade and used without further purification. All aqueous solutions were prepared using deionised water with a resistivity not less than 18.2 MΩ cm (Vivendi Water Systems). pH measurements were performed using a Hanna Instruments pH213 pH meter.

X-ray photoelectron spectroscopy (XPS) was performed using a VG clam 4 MCD analyser system, using X-ray radiation from the Al Kα band (hv=1486.7 eV). All XPS experiments were recorded using an analyser energy of 100 eV with a take-off angle of 90°. The base pressure in the analysis chamber was maintained at no more than 2.0×10⁻⁹ mbar. Each carbon powder sample was mounted on a stub using double-sided adhesive tape and then placed in the ultra-high vacuum analysis chamber of the spectrometer. To prevent samples becoming positively charged when irradiated due to emission of photoelectrons, the sample surface was bombarded with an electron beam (10 eV) from a “flood gun” within the analysis chamber of the spectrometer. Note that the peak positions reported have not been corrected relative to the C 1 s literature value of 286.6 eV to account for the effect of the flood gun on the peak positions of spectral lines. Analysis of the resulting spectra was performed using MicroCal Origin 6.0. Assignment of the spectral peaks was made using the UKSAF and NIST databases.

Combustion analysis on samples of CysOMe-carbon was carried out by determining the percentage elemental content of C, N and S using standard techniques and equipment.

### Coupling of L-cysteine Methyl Ester to Carbon Powder

Carboxyl moieties were introduced onto the graphite surface by oxidising oxygen-containing surface groups (e.g. hydroxyl and quinonyl moieties), which are known to decorate edge-plane defect sites on graphite surfaces, by stirring graphite powder in concentrated nitric acid (HNO₃) for 18 hours. The oxidised graphite powder was then washed with copious quantities of pure water until the washings ran neutral in order to remove any nitric acid from the powder sample.

Modification of graphite powder was then achieved as follows. 2 g of oxidised graphite powder was stirred in 10 cm³ of thionyl chloride (SOCl₂) for 90 minutes in order to convert the surface carboxyl groups to the corresponding acyl chloride moieties, after which time the resulting material was washed with dry chloroform to remove any unreacted thionyl chloride impurities. Next, the powder was suspended in 10 cm³ of dry chloroform containing 0.5 g of cysteine methyl ester hydrochloride. 0.27 cm³ of dry triethylamine was added to this suspension drop wise and the reaction mixture stirred at room temperature for 12 hours under an inert argon atmosphere. Finally, the resulting modified graphite powder (“CysOMe-carbon”) was washed with copious quantities of chloroform, acetonitrile acetone and pure water in order to remove any unreacted species.

### Characterisation of CysOMe-carbon Powder

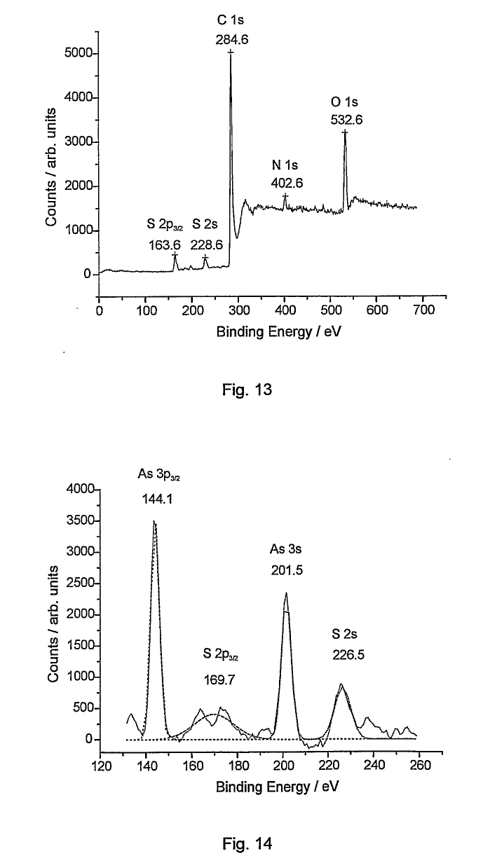

XPS was used to determine how much CysOMe had been covalently attached to the graphite surface. A sample of the CysOMe-carbon powder was mounted in the XPS spectrometer and a scan was performed from 0-1200 eV as shown in FIG. 13. Peak assignments were carried out using the UKSAF and NIST databases.

The percentage surface elemental composition was calculated from the areas under each peak in the wide spectrum adjusted by each elements individual X-ray cross-sectional area. Taking into account the relevant atomic sensitivity factors for the various elements it was found that the CysOMe comprises ca. 10% of the surface elements with a variation between different sample preparations of ±3%. This surface coverage is in good agreement with that obtained using combustion analysis which gave a surface coverage of CysOMe as being 10-14% and is approximately twice that for CysOMe-GC powder.

XPS analysis was also performed on samples of the CysOMe-carbon powder after exposure to either Cu^{II}, Cd^{II} or As^{III} solutions for sufficient times for the uptake of metal ions to be complete (see sections below). FIG. 14 shows the resulting XPS spectrum of CysOMe-carbon after exposure to As^{III} over the region where the As 3s and 3P_{3/2} and the S 2s and 2P_{3/2} spectral peaks are observed. The ratio of As^{III} to CysOMe (as measured by the sulfur spectral line areas) were found to be approximately 1:1 after taking the relative atomic sensitivity factors into account. The XPS results for the other metals studied show a similar stoichometric relationship.

### EXAMPLE 9

### Detection and Removal of Various Metal Ions Using CysOMe-carbon Powder Reagents and Equipment

Electrochemical measurements were performed using a μ-Autolab computer controlled potentiostat (EcoChemie). A three electrode cell with a solution volume of 10 cm³ was used throughout. The working electrode consisted of either a glassy carbon (GC, 3 mm diameter, BAS), a square boron doped diamond electrode (BDD, 3 mm×3 mm, Windsor Scientific Ltd) or gold (1 mm diameter, GoodFellow) macrodisc electrode. A bright platinum wire (99.99% GoodFellow) acted as the counter electrode and either a silver wire pseudo-reference electrode (99.99% GoodFellow) or a saturated calomel electrode reference electrode (SCE, Radiometer) completed the three-electrode assembly. All solutions were degassed using pure N₂ (BOC Gases) for 20 minutes prior to any electrochemical experiment being performed.

Inductively coupled plasma atomic emission spectroscopic (ICPAES) determination of As^{III} concentration in solution was analysed with the Perkin Elmer Optima 5300DV emission ICP instrument. The recommended emission wavelength was 188.979 nm and axial view is recommended for the best detection. As this is below the 200 nm threshold the optics were purged at a high flow of argon to minimise any absorption of light by water and air.

The As^{III} calibration, using 5 points (0, 50, 100, 150, 200 ppb), gave a correlation coefficient 0.9993, and the limit of detection, defined as 3 times the standard deviation of the blank, averaged from 4 blank checks each measured in 3 replicates, was found to be 9.78 ppb or 0.0098 ppm. The Perkin Elmer expected value is 1 to 10 ppb for this wavelength so the sensitivity is acceptable. The blank check solutions gave between 2.0 and 4.5 ppb for 4 checks.

### Thermodynamics and Kinetics of Cu^{II} and Cd^{II} Removal Using CysOMe-carbon Powder

The efficacy of CysOMe-carbon powder to the removal of the heavy metal ions Cu^{II}, Cd^{II} and As^{III} was determined. Concentration-time profiles were constructed for the removal of either Cu^{II} from pH 2.0 solution or Cd^{II} from pH 5.0 solution by stirring 25 mg of the modified carbon powder in 25 cm³ of solutions of varying concentration for varying amounts of time. The concentration ranges used varied between 5 μM and 500 μM, with the exact solution being determined using the LSASV analysis prior to commencing the experiments with graphite powder, and the stirring times were between 2 and 30 minutes in duration.

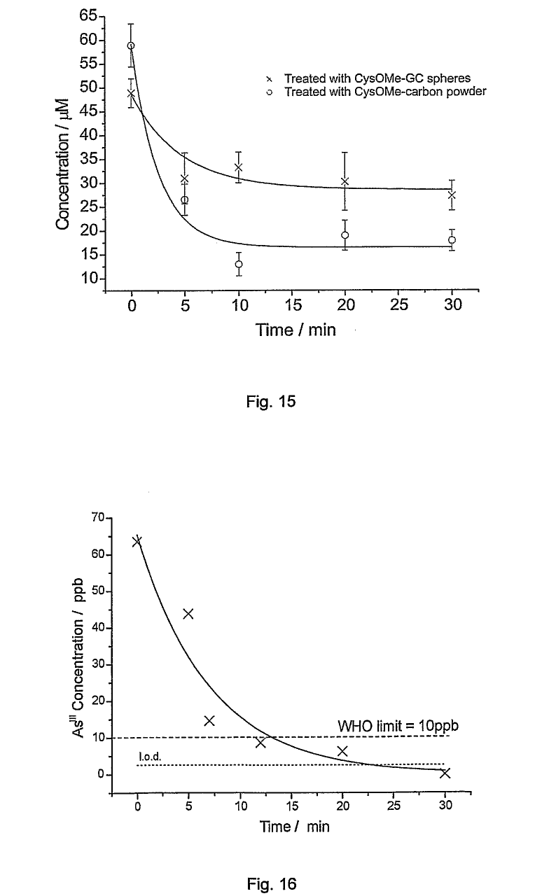

Comparison of the concentration-time profiles for the uptake of either Cu^{II} or Cd^{II} between CysOMe-carbon and CysOMe-GC demonstrates that, in each case, the modified carbon powder removed a greater amount of the metal ions in a more rapid fashion, as shown in FIG. 15. This can be attributed to the greater surface coverage of graphite powder with CysOMe than glassy carbon.

The experimental data were analysed using both the Langmuir and the Freundlich isotherm models. A comparison of the thermodynamic parameters, K′ and n, obtained for both CysOMe-carbon and CysOMe-GC for Cu^{II} and Cd^{II} uptake is given in Table 2. K′ and n are Freundlich constants relating to the maximum adsorption capacity; the larger the value of K′ and the smaller the value of n, the higher the affinity of the adsorbent towards the adsorbens.

TABLE 2

| Modified carbon powder | Metal ion | K′/L g⁻¹ | n |
|---|---|---|---|
| CysOMe-GC | Cu^{II} | 0.182 | 1.25 |
| CysOMe-carbon | Cu^{II} | 0.136 | 0.809 |
| CysOMe-GC | Cd^{II} | 0.098 | 0.90 |
| CysOMe-carbon | Cd^{II} | 0.167 | 1.18 |

The rate of metal ion adsorption by the CysOMe-carbon was determined at each concentration studied using the initial rate of metal ion adsorption from the corresponding concentration-time profile. The average adsorption rate constant, k_{ads}, of both Cu^{II} and Cd^{II} by both CysOMe-GC and CysOMe-carbon is shown in Table 3 for comparison.

TABLE 3

| Modified carbon powder | Metal ion | k_{ads}/cm s⁻¹ |
|---|---|---|
| CysOMe-GC | Cu^{II} | 2 × 10⁻⁴ |
| CysOMe-carbon | Cu^{II} | 6 × 10⁻⁴ |
| CysOMe-GC | Cd^{II} | 3 × 10⁻⁴ |
| CysOMe-carbon | Cd^{II} | 6 × 10⁻⁴ |

The faster adsorption kinetics of CysOMe-carbon powder compared to the CysOMe-GC powder reflect the increased surface coverage of CysOMe on the graphite particles, which is approximately twice that of the GC microspheres.

### Adsorption of As^{III} Ions by CysOMe-carbon Powder

The uptake of As^{III} ions by CysOMe-carbon powder was measured as follows. 40 mg of the modified carbon powder was stirred in 20 cm³ solution containing varying concentrations (10 to 150 μM) of arsenic for varying times ranging from a few minutes to several hours. The powder was then filtered off and the solution analysed using LSASV to determine the concentration of As^{III} remaining. A set of samples that had been analysed by the LSASV method were then analysed for their As^{III} concentration using ICP-AES. The results of the ICP-AES analysis were found to be in good agreement (within 5%) with those obtained by LSASV, demonstrating that the electroanalytical protocol produced accurate and reliable results.

### Removal of Trace Amounts of As^{III} Using CysMeO-carbon Powder

40 mg of CysOMe-carbon powder was stirred in 20 cm³ of a solution whose initial As^{III} concentration was determined to be ca. 70 ppb for varying times up to 30 minutes, and the concentration of As^{III} remaining in the solution monitored using the trace analysis protocol as described above.

FIG. 16 shows the resulting concentration time profile. The initial concentration of As^{III} was reduced to below the WHO limit of 10 ppb within 10 minutes of exposure to the small amount of CysOMe-carbon, and was reduced below the limit of detection of this methodology after 20 minutes of exposure.

### Determination of Cd^{III} Uptake by CysMeO-carbon Powder

The concentration of Cd^{II} remaining in a sample after exposure to CysOMe-carbon powder was determined using a LSASV protocol at a boron doped diamond electrode (BDD) developed by Banks et al (Talanta 2004, 62, 279) in pH 5.0 sodium acetate buffer. LSASV analysis was carried out using the following parameters: the BDD electrode was held at a deposition potential of −1.5 V vs. SCE for 60 seconds with stirring. The potential was then swept from −1.2 V to −0.1 V vs. SCE at a scan rate of 0.1 Vs⁻¹. A cadmium stripping peak was observed at ca. −0.8 V vs. SCE.

Prior to analysing samples with unknown concentrations of Cd^{II} the linear range was determined using the standard additions method to a sample consisting of blank acetate buffer. The results show that the LSASV analytical protocol produced a linear detection range from 1 to 20 μM with a limit of detection (based on 3σ) of 0.96 μM. Where necessary, samples were diluted prior to analysis so that their Cd^{II} concentration fell within this linear range.

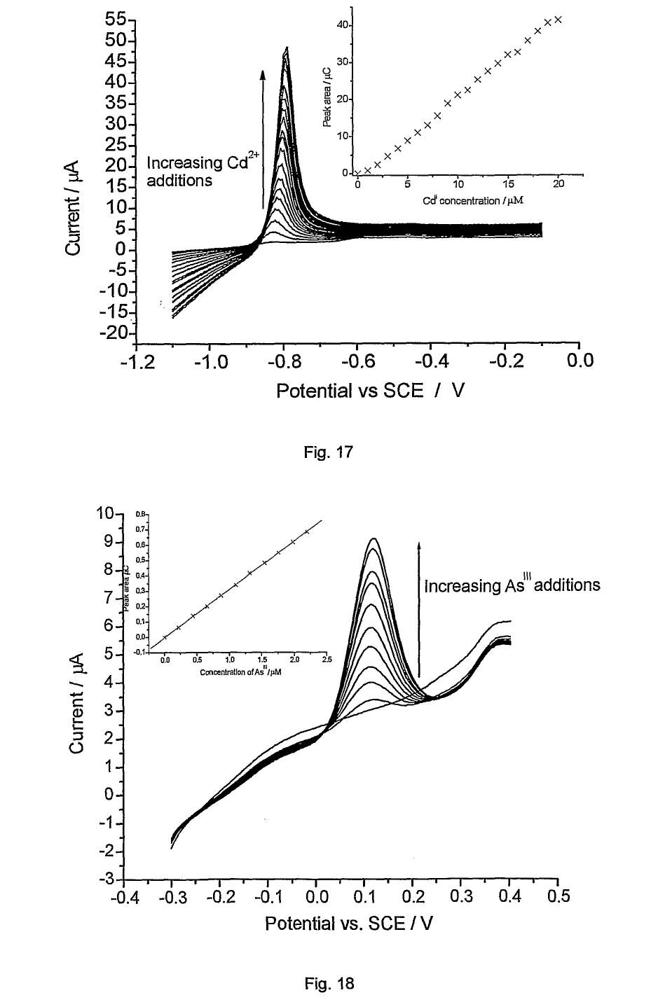

Standard 1 μM Cd^{II} additions were then added to the sample being analysed and the unknown Cd^{II} concentration was determined by constructing a standard addition plot, as shown in FIG. 17. The analysis was repeated three times and the Cd^{II} concentration remaining in the sample was calculated as the average of the three results.

### Determination of Cu^{II} Uptake by CysMeO-carbon Powder

The Cu^{II} concentration in a sample was determined using the standard addition method described above and an LSASV protocol using the following protocol. Cu^{II} analysis was performed in 0.1 M H₃PO₄, pH 2.0, using a GC working electrode and a Ag pseudo-reference electrode to avoid the formation of copper(I) chloride precipitates during the electrodeposition (which could otherwise form if a SCE reference electrode was used and are problematic for the LSASV analysis). A copper stripping peak could be observed at ca. −0.1 V vs. Ag. The linear analytical concentration range, using standard additions of 1 μM Cu^{II}, was found to be 2 to 20 μM; therefore all samples were diluted to fall within this range where necessary. LSASV was performed using a deposition potential of −1.5 V vs. Ag, deposition time 30 s, scan rate 100 mVs⁻¹ and scanning from −1.5 V to +0.8 V vs. Ag.

### Determination of As^{III} Uptake by CysMeO-carbon Powder

LSASV was performed in a solution, 10 cm³ in volume, of 0.1M HCl (pH 1.0) using a gold working electrode (diameter 1 mm) with a SCE acting as the reference electrode. The LSASV analysis was carried out on samples of relatively high concentration using the following parameters: deposition potential −0.3 V vs. SCE, deposition time 60 s with stirring for the first 5 s. Then, LSASV voltammetry was performed from −0.3 V to +0.4 V vs. SCE at 100 mVs⁻¹, step potential 5 mV. Standard 2.2 μM additions (5 μL of a 4.4 mM standard solution) were then added, and the unknown sample concentration determined form a standard addition plot. The linear range for As^{III} detection was found to be 2 to 20 μM with a limit of detection (based on the 3σ value) of 1.25 μM. Where necessary, solutions were diluted so that their concentration fell within this range prior to analysis.

For the trace analysis work, the protocol was modified slightly. The solution was stirred throughout the entire 60 s deposition time with all other parameters identical to those described above. The standard As^{III} solution was diluted so that a 5 μL aliquot added to the analysis sample corresponded to a 0.22 μM standard addition and the resulting voltammetry is shown in FIG. 18. The linear range was determined to be 0 to 2.2 μM with a limit of detection of 0.03 μM therefore it was not necessary to dilute the samples prior to analysis.

1. A derivatised carbon in which an amino acid or a derivative thereof is attached to the carbon.

2. A derivatised carbon according to claim 1, wherein the amino acid or derivative thereof is attached to carboxyl groups on said carbon.

3. A derivatised carbon according to claim 1, wherein a phenylamine group, substituted by said amino acid or derivative thereof, is attached to said carbon.

4. A derivatised carbon according to claim 1, wherein the amino acid is a sulfur-containing amino acid.

5. A derivatised carbon according to claim 4, wherein the amino acid is cysteine, glutathione, tyrosine or a derivative thereof.

6. A derivatised carbon according to claim 1, wherein the amino acid derivative is an oligomer or polymer.

7. A derivatised carbon according to claim 6, wherein the amino acid derivative is poly-S-benzyl-L-cysteine.

8. A derivatised carbon according to claim 1, wherein the carbon is graphite powder or glassy carbon spherical powder.

9. A derivatised carbon according to claim 1, wherein the carbon is glassy carbon spherical powder or pyrolytic graphite.

10. A derivatised carbon according to claim 9, wherein the carbon is glassy carbon spherical powder and the amino acid or derivative thereof is cysteine, glutathione, tyrosine or cysteamine.

11. A derivatised carbon according to claim 9, wherein the carbon is pyrolytic graphite and the amino acid or derivative thereof is polycysteine or polyglutathione.

12. A derivatised carbon according to claim 1, wherein the carbon is graphite powder or glassy carbon spherical powder and the amino acid is cysteine or a derivative thereof.

13. A derivatised carbon according to claim 12, wherein the amino acid is cysteine, cysteine methyl ester or poly-S-benzyl-L-cysteine.

14. A method of preparing a derivatised carbon in which carbon is contacted with a nitrobenzenediazonium compound under conditions such that a nitrophenyl-derivatised carbon is produced.

15. A method according to claim 14, wherein the carbon is contacted with the nitrobenzenediazonium compound in the presence of hypophosphorous acid.

16. A method according to claim 14, further comprising reducing the nitrophenyl-derivatised carbon to form an aniline-derivatised carbon.

17. A method according to claim 16, further comprising reacting the aniline-derivatised carbon with a species to produce a substituted aniline-derivatised carbon.

18. A method according to claim 17, wherein the aniline-derivatised carbon is reacted with amino acid or derivative thereof.

19. A method according to claim 18, wherein the amino acid is a sulfur-containing amino acid and the carbon is graphite powder, glassy carbon spherical powder, or pyrolytic graphite.

20. A method of preparing a derivatised carbon in which the carbon is attached directly to the amino acid or derivative thereof via carboxyl groups on the surface of the carbon, the method comprising converting carboxyl groups on the surface of the carbon to acyl halide groups and then contacting the resulting product with the amino acid or derivative thereof.

21. A method according to claim 20, wherein the acyl halide is acyl chloride.

22. A method according to claim 20, wherein the amino acid is a sulfur-containing amino acid and the carbon is graphite powder, glassy carbon spherical powder, or pyrolytic graphite.

23. A derivatised carbon according to claim 1, wherein the derivatised carbon is included in a carbon electrode.

24. A derivatised carbon according to claim 23, wherein the carbon electrode is included in an electrochemical device.

25. A method of removing metal ions from a liquid medium comprising contacting the medium with derivatised carbon according to claim 1.

26. A method according to claim 25, wherein the metal ions are selected from Cd(II), Pb(II), Zn(II), Cu (II) and As(III) ions.

27. A method of detecting the presence of metal ions in a liquid medium comprising subjecting the medium to voltammetric analysis using an electrochemical device according to claim 24.

28. A method according to claim 25, wherein the medium is an aqueous medium.
